(19) United States
(12) Patent Application Publication
(10) Pub. No.: US 2026/0256812 A1
(43) Pub. Date: Sep. 3, 2026

(54) COMPOSITION COMPRISING A COMBINATION OF A CORTICOSTEROID AND A FLAVONOID FOR USE IN TREATING EYE-RELATED DISEASES

(71) Applicant: SOUTH EAST TECHNOLOGICAL UNIVERSITY, Waterford (IE)

(72) Inventors: Madhuri DANDAMUDI, Waterford (IE); Laurence FITZHENRY, Waterford (IE); David KENT, Waterford (IE)

(73) Assignee: SOUTH EAST TECHNOLOGICAL UNIVERSITY, Waterford (IE)

(21) Appl. No.: 19/489,940

(22) PCT Filed: Jun. 7, 2024
(86) PCT No.: PCT/EP2024/065711

§ 371 Date: Dec. 4, 2025

(30) Foreign Application Priority Data
Jun. 7, 2023 (EP) 23177946.3

(51) Int. Cl. A61K 31/58; A61K 31/352; A61P 27/02

(52) U.S. Cl. CPC A61K 31/58; A61K 31/352; A61P 27/02

(57) ABSTRACT

A combination of agents for use in a method of treating an eye disorder or disease in a subject, in which the combination of agents is administered to the subject and comprises a therapeutically effective dose of a corticosteroid (triamcinolone acetonide) and a flavonol (quercetin), or a pharmaceutically acceptable analog or salt thereof.

### FIELD OF THE INVENTION

The invention relates to a composition for use in treating eye disorders. Specifically, the invention relates to a composition comprising two agents for use in treating eye disorders, such as age-related macular degeneration (AMD).

### BACKGROUND TO THE INVENTION

Posterior segment or back of the eye disorders most commonly affect the retina, choroid and optic nerves and include diseases such as retinitis pigmentosa, diabetic macular oedema, diabetic retinopathy, age-related macular degeneration (AMD), retinopathy of prematurity, etc. The primary route for delivering the therapeutic agents for the mentioned diseases is through intravitreal injections.

One example of back of the eye disease is age-related macular degeneration (AMD). AMD is a multifactorial degenerative disease with gradual loss of central vision in people aged more than 50 years. There is no cure for this disease, but treatment will delay the progression of the disease.

Audren F et al. (American Journal of Ophthalmology, vol. 142 (5), pp. 794-799 (2006)) describes a phase 2 trial of intravitreal triamcinolone acetonide for treating diffuse diabetic macular edema. Razavi M S et al. (Frontiers in Chemistry, vol. 10, pp. 850757 (2022) describes developments of a nanostructure for the ocular delivery of natural compounds, such as quercetin, which is beneficial in ocular disorders such as cataract and AMD. Hayasaka Seiji et al. (The American Journal of Chinese Medicine, vol. 40 (5), pp. 887-904 (2012)) describes the use of compounds isolated from herbs in traditional Japanese medicines for treating ocular diseases. Some of the components listed include dexamethasone and wogonin. US 2016/158320 describes a polymeric hydrogel contact lens for use in treating, ameliorating and/or stabilising posterior segment disease in the eye. The hydrogel contains an anti-inflammatory compound.

The current standard treatment for AMD is through intravitreal injections with anti-VEGF (vascular endothelial growth factor) agents. These anti-VEGF agents control choroidal neovascularization (CNV), which occurs in the late stage of AMD, where branching and development of new blood vessels evolve from the choroid, which later reaches the RPE and damages the central vision. Combination therapies like anti-VEGF agents together with photodynamic therapy and anti-VEGF agents along with corticosteroids have been used before and are known to be effective. However, these anti-VEGF intravitreal injections have serious side effects, and the monoclonal antibodies (current anti-VEGF agents) are expensive. The marketed anti-VEGF agents: abrolucizumab, aflibercept, ranibizumab, bevacizumab, faricimab-svoa, and pegaptanib sodium; can be associated with side effects such as retinal detachment, haemorrhage, an increase in intraocular pressure, etc. In addition to an increased financial burden, this treatment leads to poor patient compliance, with an estimated 1 in 4 patients not returning for follow-up treatment. As such, there is an urgent need for the development of both new and economical therapeutics.

It is an object of the present invention to overcome at least one of the above-mentioned problems.

### SUMMARY OF THE INVENTION

A novel combination of a corticosteroid (for example, triamcinolone acetonide (TA), fluocinolone acetonide, dexamethasone, prednisone, prednisolone, etc.) and a flavonol, a subclass of a flavonoid (for example, quercetin (QCN), kaempferol, myricetin, etc.) was investigated on human retinal pigment epithelial cell lines to know the potential pharmacological effect on eye conditions, such as AMD. Different disease conditions of AMD (inflammation, oxidative stress, and angiogenesis) were stimulated on human retinal cells and exposed to different concentrations of TA and quercetin both individually and in combination, in an attempt to investigate their potential for the treatment of AMD. Firstly, the individual drug concentrations and the combinations were safe on the retinal cell line and showed no signs of synergetic toxicity or changes in the morphology of the cells. The combination exhibited a better anti-inflammatory effect as the TA and QCN act majorly on different inflammatory signalling pathways (TA acts on nuclear factor kappa B and QCN on mitogen-activated protein kinase). In terms of anti-VEGF activity, both drugs act in different ways, QCN inhibits the kinase pathways leading to the deactivation of VEGF receptors, whereas TA destabilises VEGF mRNA. The combination treatments lead to the greater suppression of VEGF-C. Both the anti-oxidant assays (chemical DPPH assay and intracellular ROS DCFH-DA assay) showed similar outcomes by exhibiting the synergetic effect when treated with combination drugs (with reproducible results). QCN exhibited enhanced retinal cell migration towards the wound on its own and in combination with TA. All these findings suggest corticosteroid (for example, TA) and flavonol (for example, QCN) as a potential combination therapy to target retinal diseases such as AMD with multiple pathological conditions occurring at the same time. The drugs involved in the claimed composition are stable and can be delivered through controlled drug delivery systems, which would negate or reduce the need for intravitreal injections. There is the least possibility of in vivo toxicity or serious side effects with the claimed combination as they are already approved for human use for other conditions/purposes.

There is provided a composition for treating a retinal disease (also known as a condition or disorder) as set out in the appended claims.

There is provided a method for treating a retinal condition as set out in the appended claims.

There is provided a composition for treating a retinal condition, the composition comprising a corticosteroid and a flavonoid. Preferably, the flavonoid is a flavonol.

There is provided, as set out in the appended claims, a combination of agents for use in a method of treating an eye disorder in a subject, in which the combination of agents is administered to the subject and comprises a therapeutically effective dose of a corticosteroid and a flavonoid, or a pharmaceutically acceptable analog or salt thereof. Preferably, the flavonoid is a flavonol.

In one aspect, the corticosteroid is selected from triamcinolone acetonide, fluocinolone acetonide, dexamethasone, prednisone, methylprednisone, cortisone, hydrocortisone, and a pharmaceutically acceptable analog or salt thereof.

In one aspect, the flavonoid is selected from quercetin, kaempferol, myricetin, and a pharmaceutically acceptable analog or salt thereof. The preferred flavonoid is a flavonol.

In one aspect, the corticosteroid is triamcinolone acetonide and the flavonoid is quercetin, or a pharmaceutically acceptable analog or salt thereof. Preferably, the flavonoid is a flavonol.

In one aspect, the concentration of the corticosteroid, or pharmaceutically acceptable analog or salt thereof, is selected from about 1 μM to about 300 μM. Preferably, the concentration of the corticosteroid, or pharmaceutically acceptable analog or salt thereof, is selected from about 10 μM to about 100 μM. That is, from about 10, 15, 20, 25, 30, 35, 40, 45, 50, 55, 60, 65, 70, 75, 80, 85, 90, 95, and 100 μM. Ideally, the concentration is of the corticosteroid, or pharmaceutically acceptable analog or salt thereof, is 100 μM.

In one aspect, the concentration of the flavonoid, or pharmaceutically acceptable analog or salt thereof, is selected from 1 μM to about 75 μM. Preferably, the concentration of the flavonoid, or pharmaceutically acceptable analog or salt thereof, is selected from 1 μM to about 50 μM. That is, from about 1, 2, 3, 4, 5, 6, 7, 8, 9, 10, 15, 20, 25, 30, 35, 40, 45, and 50 μM. Preferably, the concentration of the flavonoid, or pharmaceutically acceptable analog or salt thereof, is selected from about 5 μM to about 20 μM. That is, from about 5, 6, 7, 8, 9, 10, 11, 12, 13, 14, 15, 16, 17, 18, 19, and 20 μM. Ideally, the concentration of the flavonoid, or pharmaceutically acceptable salt thereof, is 20 μM. Preferably, the flavonoid is a flavonol.

In one aspect, the wt % ratio of corticosteroid to flavonoid in the composition is about 1:0.1 to about 1:1. Preferably, the wt % ratio of corticosteroid to flavonoid in the composition is about 1:0.1 to about 1:0.3. Preferably, the flavonoid is a flavonol.

In one aspect, the combination of agents comprises about 40 μg to about 50 μg of the corticosteroid, or a pharmaceutically acceptable analog or salt thereof, and about 1 μg to about 10 μg of the flavonoid, or a pharmaceutically acceptable analog or salt thereof. Preferably, the combination of agents comprises about 43 μg of the corticosteroid, or a pharmaceutically acceptable analog or salt thereof, and about 6 μg of the flavonoid, or a pharmaceutically acceptable analog or salt thereof. Preferably, the flavonoid is a flavonol.

In one aspect, the corticosteroid is administered prior to administration of the flavonoid. In one aspect, the corticosteroid is administered following administration of the flavonoid. In one aspect, the corticosteroid and the flavonoid are administered simultaneously. Preferably, the flavonoid is a flavonol.

In one aspect, there is provided a combination of triamcinolone acetonide (a corticosteroid) and quercetin (a flavonl), or a pharmaceutically acceptable analog or salt thereof, for use in a method of treating wet age-related macular degeneration (AMD) or dry age-related macular degeneration (AMD) in a subject, in which the combination of agents is administered to the subject and comprises a therapeutically effective dose of both triamcinolone acetonide and quercetin, or a pharmaceutically acceptable analog or salt thereof.

In one aspect, the concentration of the triamcinolone acetonide, or a pharmaceutically acceptable analog or salt thereof, is selected from 1 μM to 300 μM. In one aspect, the concentration of the triamcinolone acetonide, or pharmaceutically acceptable analog or salt thereof, is selected from about 1 μM to about 300 μM. Preferably, the concentration of the triamcinolone acetonide, or pharmaceutically acceptable analog or salt thereof, is selected from about 10 μM to about 100 μM. That is, from about 10, 15, 20, 25, 30, 35, 40, 45, 50, 55, 60, 65, 70, 75, 80, 85, 90, 95, and 100 μM. Ideally, the concentration is of the triamcinolone acetonide, or pharmaceutically acceptable analog or salt thereof, is 100 μM.

In one aspect, the concentration of the quercetin, or pharmaceutically acceptable analog or salt thereof, is selected from 1 μM to 75 μM. Preferably, the concentration of the quercetin, or pharmaceutically acceptable analog or salt thereof, is selected from 1 μM to about 50 μM. That is, from about 1, 2, 3, 4, 5, 6, 7, 8, 9, 10, 15, 20, 25, 30, 35, 40, 45, and 50 μM. Preferably, the concentration of the quercetin, or pharmaceutically acceptable analog or salt thereof, is selected from about 5 μM to about 20 μM. That is, from about 5, 6, 7, 8, 9, 10, 11, 12, 13, 14, 15, 16, 17, 18, 19, and 20 μM. Ideally, the concentration of the quercetin, or pharmaceutically acceptable analog or salt thereof, is 20 μM.

In one aspect, the wt % ratio of triamcinolone acetonide to quercetin to in the composition is 1:0.1 to 1:1. Preferably, the wt % ratio of triamcinolone acetonide to quercetin in the composition is 1:0.1 to 1:0.3.

In one aspect, the combination of triamcinolone acetonide and quercetin, or pharmaceutically acceptable analogs or salts thereof, comprises 40 μg to 50 μg of triamcinolone acetonide, or pharmaceutically acceptable analog or salt thereof, and 1 μg to 10 μg of quercetin, or pharmaceutically acceptable analog or salt thereof. Preferably, the combination of triamcinolone acetonide and quercetin comprises about 43 μg of the triamcinolone acetonide, or a pharmaceutically acceptable analog or salt thereof, and about 6 μg of the quercetin, or a pharmaceutically acceptable analog or salt thereof.

In one aspect, the triamcinolone acetonide is administered prior to administration of the quercetin. In one aspect, the triamcinolone acetonide is administered following administration of the quercetin. In one aspect, the triamcinolone acetonide and the quercetin are administered simultaneously.

In one aspect, the combination of triamcinolone acetonide, or a pharmaceutically acceptable analog or salt thereof, and quercetin, or a pharmaceutically acceptable analog or salt thereof, further comprises a pharmaceutically acceptable carrier.

In one aspect, the corticosteroid and the flavonoid are administered orally or parenterally. Preferably, the parenteral route of administration is selected from intravenous, intramuscular, intraocular, subcutaneous, transdermal, via airway (aerosol), pulmonary, nasal, rectal, and topical administration. Preferably, the route of administration is selected from injection, infusion, instillation, inhalation, or ingestion. Preferably, the flavonoid is a flavonol.

In one aspect, the combination of agents described above, for use as described above, further comprises a pharmaceutically acceptable carrier.

In one aspect, the eye disorder is selected from wet age-related macular degeneration (AMD), dry age-related macular degeneration (AMD), retinitis pigmentosa (RP), Behçet's disease, blepharitis, central retinal vein occlusion (CRVO), diabetic retinopathy, diabetic macular edema (DME), neovascular glaucoma, macular edema, uveitis, retinal vein occlusion, ocular histoplasmosis syndrome (OHS), retinopathy of prematurity (ROP), and the like. Preferably, the eye disorder is wet or dry age-related macular degeneration.

In one aspect, there is provided a pharmaceutical composition comprising a corticosteroid and a flavonoid, or a pharmaceutically acceptable salt or analog thereof, and a pharmaceutically acceptable excipient. Preferably, the flavonoid is a flavonol.

In one aspect, the pharmaceutical composition is in the form of an eye drop, an intravitreal injection, a suprachoroidal injection, a retrobulbar injection, a subretinal injection, a sub-tenon injection, and a peribulbar injection.

In one aspect, there is provided a kit of parts comprising one or more doses of a corticosteroid, or a pharmaceutically acceptable salt or analog thereof, and one or more doses of a flavonoid, or a pharmaceutically acceptable salt or analog thereof. Preferably, the flavonoid is a flavonol.

In one aspect, there is provided a method for treating an eye disorder, the method comprising administering a therapeutic amount of a corticosteroid and a flavonoid, or pharmaceutically acceptable salts or analogs thereof, to the eye of a subject in need thereof. Preferably, the flavonoid is a flavonol.

In one aspect, there is provided a method for alleviating a symptom of an eye disorder, the method comprising administering a therapeutic amount of a corticosteroid and a flavonoid, or pharmaceutically acceptable salts or analogs thereof, to the eye of a subject in need thereof. Preferably, the flavonoid is a flavonol.

In one aspect of the methods, the corticosteroid is selected from triamcinolone acetonide, fluocinolone acetonide, dexamethasone, loteprednol etabonate, prednisone, methylprednisone, cortisone, hydrocortisone, and a pharmaceutically acceptable analog or salt thereof.

In one aspect of the methods, the flavonoid is selected from quercetin, kaempferol, myricetin and a pharmaceutically acceptable analog or salt thereof. The flavonoids presented here are flavonols.

In one aspect of the methods, the corticosteroid is triamcinolone acetonide and the flavonoid is a flavonol, quercetin, or pharmaceutically acceptable analogs or salts thereof.

In one aspect of the methods, the concentration of the corticosteroid, or a pharmaceutically acceptable analog or salt thereof, is selected from about 1 μM to about 300 μM. Preferably, the concentration of the corticosteroid, or a pharmaceutically acceptable analog or salt thereof, is selected from about 10 μM to about 100 μM. That is, from about 10, 15, 20, 25, 30, 35, 40, 45, 50, 55, 60, 65, 70, 75, 80, 85, 90, 95, and 100 μM. Ideally, the concentration is of the corticosteroid, or pharmaceutically acceptable analog or salt thereof, is 100 μM.

In one aspect of the methods, the concentration of the flavonoid, or a pharmaceutically acceptable analog or salt thereof, is selected from about 1 μM to about 75 μM.

Preferably, the concentration of the flavonoid, or pharmaceutically acceptable analog or salt thereof, is selected from 1 μM to about 50 μM. That is, from about 1, 2, 3, 4, 5, 6, 7, 8, 9, 10, 15, 20, 25, 30, 35, 40, 45, and 50 μM. Preferably, the concentration of the flavonoid, or pharmaceutically acceptable analog or salt thereof, is selected from about 5 μM to about 20 μM. That is, from about 5, 6, 7, 8, 9, 10, 11, 12, 13, 14, 15, 16, 17, 18, 19, and 20 μM. Ideally, the concentration of the flavonoid, or pharmaceutically acceptable salt thereof, is 20 μM. Preferably, the flavonoid is a flavonol.

In one aspect of the methods, the wt % ratio of corticosteroid to flavonoid in the composition is about 1:0.1 to about 1:1. Preferably, the wt % ratio of corticosteroid to flavonoid in the composition is about 1:0.1 to about 1:0.3. Preferably, the flavonoid is a flavonol.

In one aspect of the methods, the composition comprises about 40 μg to about 50 μg of the corticosteroid, or a pharmaceutically acceptable analog or salt thereof, and about 1 μg to about 10 μg of the flavonoid, or a pharmaceutically acceptable analog or salt thereof. Preferably, the composition comprises 43 μg of the corticosteroid, or a pharmaceutically acceptable analog or salt thereof, and about 6 μg of the flavonoid, or a pharmaceutically acceptable analog or salt thereof. Preferably, the flavonoid is a flavonol.

In one aspect of the methods, the corticosteroid is administered prior to administration of the flavonoid. In one aspect of the methods, the corticosteroid is administered following administration of the flavonoid. In one aspect of the methods, the corticosteroid and the flavonoid are administered simultaneously. Preferably, the flavonoid is a flavonol.

In one aspect of the methods, the corticosteroid and the flavonoid are administered orally or parenterally. Preferably, the parenteral route of administration is selected from intravenous, intramuscular, intraocular, subcutaneous, transdermal, via airway (aerosol), pulmonary, nasal, rectal, and topical administration. Preferably, the flavonoid is a flavonol.

In one aspect of the methods, the route of administration is selected from injection, infusion, instillation, inhalation, or ingestion. In one aspect, the route of administration is in the form of an eye drop, an intravitreal injection, a suprachoroidal injection, a retrobulbar injection, a subretinal injection, a sub-tenon injection, and a peribulbar injection.

In one aspect of the methods, the composition used in the methods further comprises a pharmaceutically acceptable carrier.

In one aspect of the methods, the eye disorder is selected from wet age-related macular degeneration (AMD), dry age-related macular degeneration (AMD), retinitis pigmentosa (RP), Behçet's disease, blepharitis, central retinal vein occlusion (CRVO), diabetic retinopathy, diabetic macular edema (DME), neovascular glaucoma, macular edema, uveitis, retinal vein occlusion, ocular histoplasmosis syndrome (OHS), retinopathy of prematurity (ROP), and the like. Preferably, the eye disorder or disease is wet or dry age-related macular degeneration.

### Definitions

In the specification, the term “flavonoid” should be understood to mean a class of polyphenolic secondary metabolites found in plants. Chemically, flavonoids have the general structure of a 15-carbon skeleton, which consists of two phenyl rings (A and B) and a heterocyclic ring (C, the ring containing the embedded oxygen). This carbon structure can be abbreviated C6-C3-C6. They can be classified into: flavonoids or bioflavonoids; isoflavonoids, derived from 3-phenylchromen-4-one (3-phenyl-1,4-benzopyrone) structure; flavonols, a subclass of flavonoid having the 3-hydroxyflavone backbone; and neoflavonoids, derived from 4-phenylcoumarin (4-phenyl-1,2-benzopyrone) structure. Examples of flavonols include azaleatin, fisetin, galangin, gossypetin, kaempferide, isorhamnetin, morin, natsudaidain, pachypodol, rhamnazin, thamnetin, quercetin, kaempferol, myricetin, and isomers or analogs thereof. When the term “flavonoid” is used in the description of this specification, it should be interpreted as referring to a flavonol, a subclass of flavonoid. The flavonols used herein are naturally occurring.

In the specification, the term “corticosteroid” should be understood to mean an anti-inflammatory agent that closely resemble the natural hormone cortisol, produced by the adrenal gland. Two main classes of corticosteroids, glucocorticoids and mineralocorticoids, are involved in a wide range of physiological processes, including stress response, immune response, and regulation of inflammation, carbohydrate metabolism, protein catabolism, blood electrolyte levels, and behavior. Examples of synthetic and natural corticosteroids include triamcinolone acetonide (TA), amcinonide, budesonide, desonide, fluocinolone acetonide, fluocinonide, halcinonide, cortisone, prednisone, hydrocortisone, ciclesonide, cortisone acetate, hydrocortisone aceponate, hydrocortisone acetate, hydrocortisone buteprate, hydrocortisone butyrate, hydrocortisone valerate, prednicarbate, tixocortol pivalate, aldosterone, prednisone, methylprednisone, dexamethasone, and isomers or analogs thereof.

In the specification, the term “eye disorder”, “eye disease”, “disorders of the eye”, or “diseases of the eye”, which all can be used interchangeably, should be understood to mean conditions specific to the eye and components thereof. Such conditions include wet age-related macular degeneration (AMD), dry age-related macular degeneration (AMD), retinitis pigmentosa (RP), Behcet's disease, blepharitis, central retinal vein occlusion (CRVO), diabetic retinopathy, diabetic macular edema (DME), neovascular glaucoma, macular edema, uveitis, retinal vein occlusion, ocular histoplasmosis syndrome (OHS), retinopathy of prematurity (ROP), and the like.

In the specification, the term “healthy” should be understood to mean where the individual or patient has no underlying medical condition, infection, inflammatory response, condition or otherwise occurring.

In the specification, the term “inflammatory condition” should be understood to mean immune-related conditions resulting in allergic reactions, myopathies and abnormal inflammation and non-immune related conditions having causal origins in inflammatory processes. Examples include retinal inflammation (or retinal inflammation at the back of the eye), and the like.

In the specification, the term “individual” or “patient” should be understood to mean all mammals, for example, a human, primates, non-human primates, farm animals (such as pigs, horses, goats, sheep, cows (including bulls, bullocks, heifers etc.), donkey, reindeer, etc.), veterinary mammals (such as dogs, cats, rabbits, hamsters, guinea pigs, mice, rats, ferrets, etc.), and mammals kept in captivity (such as lions, tigers, elephants, zebras, giraffes, pandas, rhino, hippopotamus, etc.), and other mammals and higher mammals for which the use of the invention is practicable.

In this specification, the term “biological sample” should be understood to mean aqueous humour, vitreous humour, blood or blood derivatives (serum, plasma, etc.), urine, saliva or cerebrospinal fluid.

In the specification, the term “treatment” should be understood to mean prohibiting, preventing, restraining, and slowing, stopping or reversing progression or severity of a condition associated with the eye.

In the specification, the term “administer” or “administering” to a subject should be understood mean providing the agents in pharmaceutically acceptable compositions. These pharmaceutically acceptable compositions comprise a therapeutically-effective amount of a combination of a corticosteroid and a flavonoid, formulated together with one or more pharmaceutically acceptable carriers (additives) and/or diluents. The pharmaceutical compositions can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin or eye or eye area; (4) intravaginally or intrarectally, for example, as a pessary, cream or foam; (5) sublingually; (6) ocularly; (7) transdermally; (8) transmucosally (e.g. as a nasal spray or suppository); or (9) nasally. Additionally, agents can be implanted into a patient or injected using a drug delivery system. See, for example, Urquhart, et al., Ann. Rev. Pharmacol. Toxicol. 24:199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960. Guidance for formulations can be found in e.g. Remington: The Science and Practice of Pharmacy by Alfonso R. Gelmaro (Ed.) 20th edition: Dec. 15, 2000, Lippincott, Williams $ Wilkins, ISBN: 0683306472, and are briefly described below.

As used herein, the term “administer” or “administering” refers to the placement of a composition into a subject by a method or route which results in at least partial localization of the composition at a desired site such that desired effect is produced. An agent or composition described herein can be administered by any appropriate route known in the art including, but not limited to, oral or parenteral routes, including intravenous, intramuscular, intraocular, subcutaneous, transdermal, airway (aerosol), pulmonary, nasal, rectal, and topical (including ocular, buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intraocular, intracardiac, intradermal, intraperitoneal, transtracheal, subcutaneous, subcuticular, intraarticular, subcapsular, subarachnoid, intraspinal, intracerebro spinal, and intrasternal injection and infusion.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatine; (7) lubricating agents, such as magnesium stearate, sodium lauryl sulphate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerine, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; (24) C2-C12 alcohols, such as ethanol; and (26) other non-toxic compatible substances employed in pharmaceutical formulations. Wetting agents, colouring agents, release agents, coating agents, sweetening agents, flavouring agents, perfuming agents, preservative and antioxidants can also be present in the formulation. The amount of agent which can be combined with a carrier material to produce a single dosage form will generally be that amount of the agent which produces a therapeutic effect. Generally out of one hundred percent, this amount will range from about 0.1% to 99% of agent, preferably from about 5% to about 70%, most preferably from 10% to about 30%.

Formulations suitable for parenteral administration conveniently include sterile aqueous preparation of the active agent which is preferably isotonic with the blood of the recipient. Thus, such formulations may conveniently contain distilled water, 5% dextrose in distilled water or saline. Useful formulations also include concentrated solutions or solids containing the agent which upon dilution with an appropriate solvent give a solution suitable for parental administration above.

For enteral administration, an agent can be incorporated into an inert carrier in discrete units such as capsules, cachets, tablets or lozenges, each containing a predetermined amount of the active agent; as a powder or granules; or a suspension or solution in an aqueous liquid or non-aqueous liquid, e.g., a syrup, an elixir, an emulsion or a draught. Suitable carriers may be starches or sugars and include lubricants, flavourings, binders, and other materials of the same nature.

A tablet may be made by compression or moulding, optionally with one or more accessory ingredients. Compressed tablets may be prepared by compressing in a suitable machine the active agent in a free-flowing form, e.g., a powder or granules, optionally mixed with accessory ingredients, e.g., binders, lubricants, inert diluents, surface active or dispersing agents. Moulded tablets may be made by moulding in a suitable machine, a mixture of the powdered active agent with any suitable carrier.

A syrup or suspension may be made by adding the active agent to a concentrated, aqueous solution of a sugar, e.g., sucrose, to which may also be added any accessory ingredients. Such accessory ingredients may include flavouring, an agent to retard crystallization of the sugar or an agent to increase the solubility of any other ingredient, e.g., as a polyhydric alcohol, for example, glycerol or sorbitol.

Formulations for rectal administration may be presented as a suppository with a conventional carrier, e.g., cocoa butter or Witepsol S55 (trademark of Dynamite Nobel Chemical, Germany), for a suppository base.

Formulations for oral administration may be presented with an enhancer. Orally-acceptable absorption enhancers include surfactants such as sodium lauryl sulphate, palmitoyl carnitine, Laureth-9, phosphatidylcholine, cyclodextrin and derivatives thereof; bile salts such as sodium deoxycholate, sodium taurocholate, sodium glycocholate, and sodium fusidate; chelating agents including EDTA, citric acid and salicylates; and fatty acids (e.g., oleic acid, lauric acid, acylcarnitines, mono- and diglycerides). Other oral absorption enhancers include benzalkonium chloride, benzethonium chloride, CHAPS (3-(3-cholamidopropyl)-dimethylammonio-1-propanesulfonate), Big-CHAPS (N, N-bis(3-D-gluconamidopropyl)-cholamide), chlorobutanol, octoxynol-9, benzyl alcohol, phenols, cresols, and alkyl alcohols. In one embodiment, the oral absorption enhancer may be sodium lauryl sulphate.

The terms “decrease”, “reduced”, “reduction”, or “inhibit” are all used herein generally to mean a decrease by a statistically significant amount. However, for avoidance of doubt, “reduced”, “reduction”, “decrease”, or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90%. In one embodiment, there is a 100% decrease (e.g. absent level as compared to a reference sample).

The terms “increased”, “increase”, “enhance”, or “activate” are all used herein to generally mean an increase by a statically significant amount; for the avoidance of any doubt, the terms “increased”, “increase”, “enhance”, or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### BRIEF DESCRIPTION OF THE DRAWINGS

The invention will be more clearly understood from the following description of an embodiment thereof, given by way of example only, with reference to the accompanying drawings, in which:

FIG. 1 is a bar chart illustrating % cell viability of (a) TA and (b) QCN for a range of concentrations between 10 and 250 μM, data points represent the average±SD of n=3 biological replicates for (a) and technical replicates for (b).

FIG. 2 is a bar chart showing % cell viability of QCN for a range of concentrations between 1 and 100 μM, n=3±SD.

FIG. 3 is a bar chart showing % cell viability a combination of drugs (triamcinolone acetonide and QCN, TA+Q) on ARPE-19 cells, n=3±SD.

FIG. 4 is a bar chart showing an assessment of cytotoxicity of (a) LPS and (b) hydrogen peroxide on ARPE-19 cells up to 48 h, n=3±SD.

FIG. 5 is a bar chart showing the secretion of IL-6 by ARPE-19 on stimulation with different concentrations of (a) LPS and (b) hydrogen peroxide, n=3±SD.

FIG. 6 is a bar chart showing the levels of IL-8 expressed by ARPE-19 upon stimulation with LPS, n=2±SD.

FIG. 7 is a bar chart showing the results of an investigation into the anti-inflammatory effect of TA and QCN individually and in combination using IL-6 ELISA: ARPE-19 cells were stimulated using 10 μg/mL LPS for 24 hr followed by 24 hr treatments. ** P<0.01 (highly significant) * P<0.05 (significant), n=2±SD.

FIG. 8 is a bar chart showing the results of an investigation into the anti-inflammatory effect of TA and QCN on their own and in combination using IL-8 ELISA: ARPE-19 cells were stimulated using 10 μg/mL LPS for 24 hr followed by 24 hr treatments. *** P<0.001 (very highly significant) ** P<0.01 (highly significant) * P<0.05 (significant), n=2±SD.

FIG. 9 is a bar chart showing the results of an investigation into the anti-inflammatory effect of TA and QCN on their own and in combination using MCP-1 ELISA: ARPE-19 cells were stimulated using 10 μg/mL LPS for 24 hr followed by 24 hr treatments. *** P<0.001 (very highly significant) ** P<0.01 (highly significant) * P<0.05 (significant), n=2±SD.

FIG. 10 is a bar chart showing the results of an investigation into the anti-VEGF activity of TA and QCN on their own and in combination using VEGF-C ELISA: ARPE-19 cells were stimulated using 10 μg/mL LPS for 24 hr followed by 24 hr treatments. *** P<0.001 (very highly significant) ** P<0.01 (highly significant) * P<0.05 (significant), n=2±SD.

FIG. 11 is a bar chart showing the results of an investigation into the antioxidant activity using a DPPH assay by considering the % inhibition of DPPH free radical agent. * P<0.05 (significant), n=2±SD.

FIG. 12 shows the flow cytometer analysis of ROS generation using DCFH-DA dye (a) unstimulated stained cells (b) stimulated stained cells with 300 M hydrogen peroxide.

FIG. 13 shows the comparison of unstimulated (a) stained and (b) unstained ARPE-19 cells using flow cytometer analysis.

FIG. 14 is a bar chart showing the investigation of intracellular ROS levels by estimating mean fluorescence intensity (MFI) using flow cytometer.) ** P<0.01 (highly significant) in comparison with control stimulated stained cells. n=3±SD.

FIG. 15 shows fluorescent ARPE-19 cells in gate D4 and the non-fluorescent cells in gate D3 in flow cytometer analysis, where: (a) control stimulated (b) TA 75 μM (c) TA 100 μM (d) Q 15 μM (e) Q 20 μM (f) TA 75+Q 15 μM (g) TA 75+Q 20 μM (h) TA 100+Q 15 μM (i) TA 100+Q 20 μM.

FIG. 16 shows examination of stained ROS species by mean fluorescence intensity (MFI) measured using flow cytometry. (a) control stimulated (b) TA 75 μM (c) TA 100 μM (d) Q 15 μM (e) QCN 20 μM (f) TA 75+QCN 15 μM (g) TA 75+QCN 20 μM (h) TA 100+QCN 15 μM (i) TA 100+QCN 20 μM.

FIG. 17 is a bar chart showing an assessment of cell migration using scratch assay at different time points. n=3±SD.

FIG. 18 shows microscopic pictures representing the wound closure process of the ARPE-19 cells treated with TA 100+Q 20 μM at different time points (0 hr, 3 hr, 6 hr, 9 hr, 24 hr).

FIG. 19 shows microscopic pictures representing the wound closure process of the ARPE-19 cells treated with TA 100 μM at different time points (0 hr, 3 hr, 6 hr, 9 hr, 24 hr, 27 hr, 30 hr).

### DETAILED DESCRIPTION OF THE DRAWINGS

### Materials and Methods

Adult retinal pigment epithelium-19 (CRL-2302™) and Fetal Bovine Serum (ATCC-30-2025, EU Approved, South American Origin) were purchased from LGC standards Ltd, UK. DMEM/F-12 cell culture media (Gibco™ 31330038), Trypsin (Gibco™ 25300054), Penicillin-Streptomycin (Gibco™ 15070063) were procured from Fisher Scientific, Ireland. p-nitrophenyl phosphate (PNPP, CatLog #34045) was purchased from ThermoFisher Scientific, Ireland. IL-6, IL-8, MCP-1, and VEGF-C enzyme-linked immunosorbent assay (ELISA) kits were purchased from Assay genie, Ireland. 96, 24 and 6 well plates and serological pipettes were procured from Greiner bio-one, Cruinn diagnostics, Ireland.

The following equipment was used throughout the experimental work: Refrigerated centrifuge (Sigma 3-18KS, Focus scientific, Ireland), plate reader (HTS Plate Reader-MSD Model 1250 Sector Imager, U.S), inCu safe cell culture incubator (Davidson and Hardy Ltd, Ireland), −80° C. chest freezer (Medical supply company, Ltd, Ireland), flow cytometer (Cytomics FC500, Beckman Coulter, United States), Memmert—Water Bath (Mason technology, Ireland), Airstream® Class II Biological Safety Cabinet (ESCO, Mason technology, Ireland), and Classic Vortex Mixer (Fisherbrand™, Ireland).

### Cell Culture

ARPE-19 (adult retinal pigment epithelium-19) cell line was cultured using cell culture media containing a 1:1 mixture of DMEM and F-12 nutrient mixture. The media was supplemented with 10% FBS and 1% penicillin-streptomycin antibiotic mixture. During the culture, cells were maintained at 37° C. with 5% CO₂ in a humidified incubator and sub-cultured with trypsin upon confluency.

### Cytotoxicity Evaluation of Individual Drugs and Drug Combinations

Cytotoxicity evaluation of the drugs, both individually and in combination, was performed using the acid phosphatase assay (APA). The cytotoxicity assay was performed on TA and QCN from 5 to 250 μM and 1 to 250 μM, respectively. After investigating individual drugs, drug combinations were tested as outlined in Table 1.

TABLE 1
Concentrations of QCN in combination with
TA tested for cytotoxicity on ARPE-19.

| TA (μM) | Quercetin (μM) |
|---|---|
| 10 | 5, 10, 15, 20 |
| 25 | 5, 10, 15, 20 |
| 50 | 5, 10, 15, 20 |
| 75 | 5, 10, 15, 20 |
| 100 | 5, 10, 15, 20 |

Initially ARPE-19 cells were seeded onto a 96-well plate at a seeding density of 5000 cells/well containing 100 μL of cell culture media and cultured for 24 hr. After 24 hr, treatments were added to the wells and incubated for the period of the study (24 h and 48 hr). After the treatment period, media was removed, and cells were washed twice with PBS. Upon washing, 100 μL of 10 mM PNPP substrate dissolved in 0.1 M sodium acetate buffer was added to the wells and incubated for 2 hr. Finally, 50 μL of stop solution (1 M sodium hydroxide) was added and the plate was analysed in the plate reader at 405 nm.

### Anti-Inflammatory and Anti-VEGF Activities on ARPE-19 Using ELISA

The human IL-6, IL-8, MCP-1, and VEGF-C ELISA kits were used according to the manufacturer's protocol to investigate the cytokine secretions in the cell supernatants. Cells were seeded onto a 24-well plate at a seeding density of 3×10⁴ cells/well with 500 μL of cell culture media and cultured for 24 hr. Cells were kept overnight in low serum media (with 1% FBS) to synchronise their growth phase before stimulating inflammation. After 24 hr cells were stimulated with 10 μg/mL LPS to induce inflammation for a duration of 24 hr. Upon stimulation, cells were exposed to various treatments of drugs alone and in combination for 24 hr. After the treatment duration media conditioned by treated cells were collected and analysed for the cytokines and VEGF-C secretions using the ELISA kits. ELISA was performed according to the manufacturer's protocol.

### Scratch Assay

For the scratch or wound healing assay cells were seeded onto six-well plates with a seeding density of 25×10⁴ cells/well. Upon reaching 80-90% confluency, a sterile 200 μL pipette tip was used to scrape the confluent monolayer of cells in a horizontal line. After creating the scratch, cell debris was washed with PBS and the cell culture media containing treatments was added to each well. The scratch was photographed using an Olympus EP50 microscope at 10× magnification at 0, 6, 9, 24, 27 and 30 h. “Image J” software was used to analyse the width of the scratch.

### Antioxidant Activity

### DPPH (2,2-Diphenyl-1-Picryl-Hydrazyl-Hydrate) Assay

The DPPH assay was performed in a 96-well plate where 20 μL of methanolic DPPH solution was added to 180 μL of methanolic solutions of treatments. Based on the outcome from anti-inflammatory studies, the higher concentrations of TA and QCN, both individually and in combination were investigated for antioxidant activity (TA 75, 100 μM and QCN 15, 20 μM). This reaction mixture was incubated for 30 min in darkness at room temperature and the absorbance was measured at 517 nm using a microplate reader.

### DCFH-DA (Dichloro-Dihydro-Fluorescein Diacetate) Assay

ARPE-19 cells were seeded onto 6-well plates at a seeding density of 25×10⁴ cells/well and incubated for 24 hr. At the 24 hr time point cells were stimulated with stimulants to induce oxidative stress. The stimulants, LPS and hydrogen peroxide (H₂O₂) were tested at concentrations 10, 20, 40 μg/mL and 100, 200, 300 μM, respectively (H₂O₂, 300 μM proved to be an effective stimulant). Following stimulation cells were exposed to similar concentrations of TA and QCN as used for the DPPH assay (both individually and in combination) for 24 hr and after the treatment period cells were incubated with 20 μM DCFH-DA dye. Upon incubation with dye, cells were detached using trypsin and resuspended in PBS for analysis using flow cytometry.

### Statistics

Statistical differences between the treatments were assessed using student's T test. The results were considered statistically significant if the P value was less than 0.05. All statistical analysis were performed using both excel and the GraphPad® software.

### Results and Discussion

### Drug Cytotoxicity Study

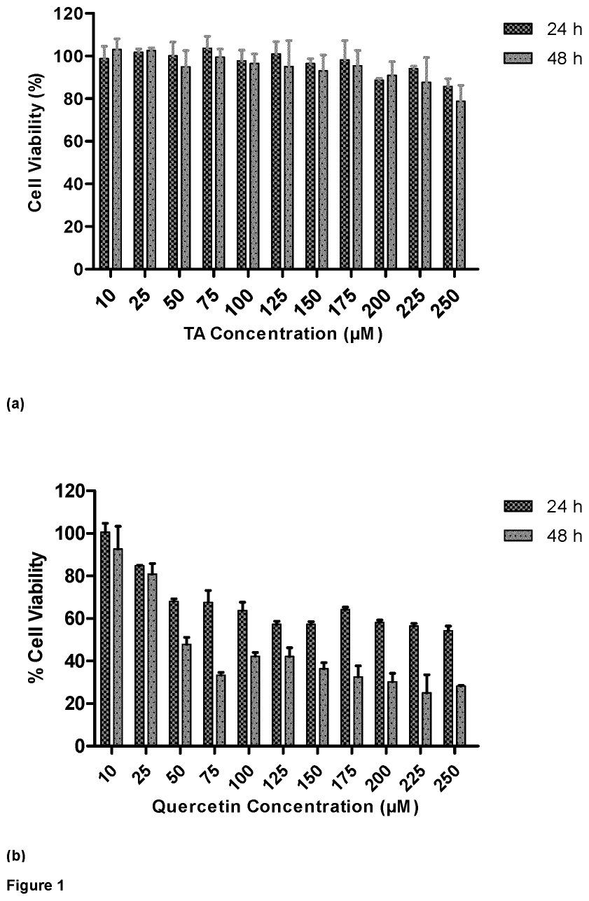

Before investigating the combination drugs on ARPE-19 cells, individual drugs were studied. The concentrations were selected from previously published studies (higher concentrations were included to establish safe and toxic concentrations). Both TA and QCN were evaluated between 10 and 250 μM concentrations for 24 hr and 48 hr periods (FIG. 1(a), 1(b)). The viability of cells was not affected with exposure to TA from 10 to 250 μM and no changes in cell morphology was observed.

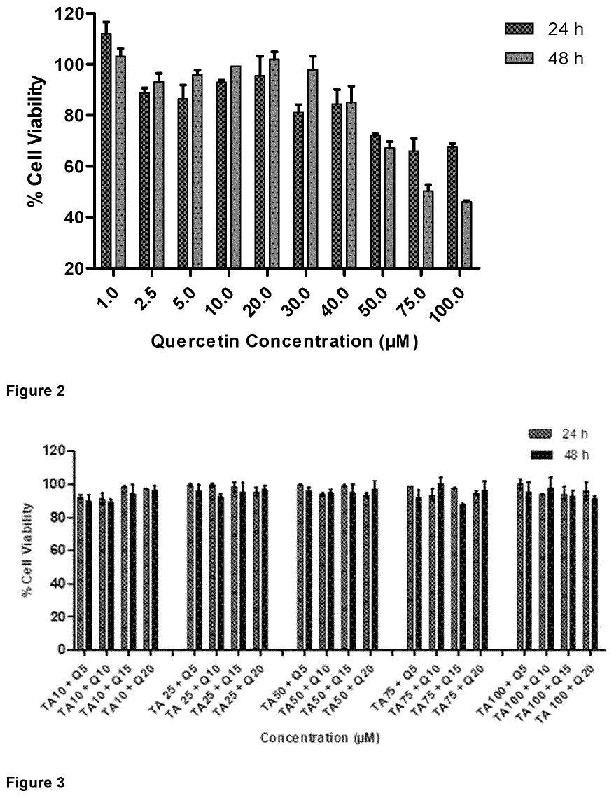

Unlike TA in FIG. 1(a), QCN exhibited a decrease in cell viability with increase in concentration as depicted in FIG. 1(b). The QCN concentration up to 25 μM displayed more than 80% cell viability but the higher concentrations exhibited a toxic effects on cells. Given these results, a further study focused on lower concentrations of QCN, 1-100 μM. The QCN concentrations from 1 to 20 μM displayed more than 90% cell viability (FIG. 2) and proved to be safe on retinal cells. Based on these results and effective concentrations of the drugs on the pathology of AMD in previous studies; TA 10, 25, 50, 75 and 100 μM and QCN from 5, 10, 15 and 20 μM was chosen to be studied in combination.

Drug combinations as mentioned above showed no synergetic toxicity on ARPE-19 cells or changes to cell morphology (FIG. 3). The cytotoxicity assay estimates the loss of cellular and intercellular functions or structure, which includes cytotoxicity effects. Hence, this provides insight into any potential tissue/cell injury and irritation during application. This in vitro cytotoxicity study suggests that the individual drugs and in combination were safe on human retinal cell lines.

### Anti-Inflammatory Activity Studies Using ARPE-19 Cells

Multiple studies on biological samples from AMD patients and pathophysiological studies supports the conclusion that AMD is a multifactorial disorder involving RPE dysfunction and damage to photoreceptor cells (mainly due to inflammatory conditions and oxidative stress). RPE play a crucial role in the formation of the blood-retinal barrier (BRB), establishment of ocular immune privilege and in secretion of immunomodulatory factors to monitor immunogenic inflammation. In the case of AMD, damage to RPE effects the ocular immune tolerance distorting BRB, downregulating the immune and anti-inflammatory proteins resulting in attack by T cells on autoantigens. This process leads to stimulation of inflammatory mediators and pathways resulting in the production of cytokines and chemokines, such as IL-4, 5, 6, 8, 10, 13, 17, TGF-beta, IFN-Y, MCP-1, and VEGF. Various inflammatory cytokines were reported to be raised in the serum, fluids, systematically, or in the ocular tissues of AMD patients. Investigations of blood samples of the AMD patients showed elevated levels of monocyte chemotactic protein-1 (MCP-1), IL-6 and IL-8 and that monocytes may lead to the progression of AMD. Considering the previous findings and their role in AMD inflammation, IL-6, IL-8, and MCP-1 were prioritized for the current study. Suppression or inhibition of these cytokines and mediators is the key indicator for study of the anti-inflammatory properties of the chosen drug or treatment.

To induce inflammation, two stimulants were screened; lipopolysaccharide (LPS) and hydrogen peroxide (H₂O₂), which had worked effectively in previous studies. LPS is the molecule abundantly available on cell membranes of gram-negative bacteria that can cause inflammatory events by stimulating the release of several cytokines in a vast number of cell types. LPS binds to the CD14 receptor, which exists as membrane protein on ARPE-19 cells leading to the activation of Toll-like-receptors (TLRs) pathways resulting in secretion of inflammatory cytokines. H₂O₂ elevates the intracellular ROS resulting in oxidative stress disrupting cellular balance leading to chronic inflammation.

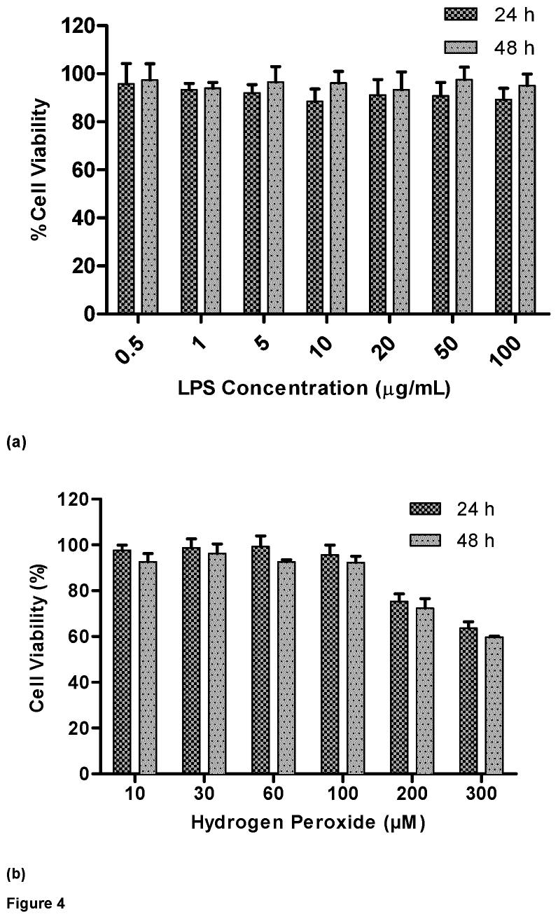

To investigate the anti-inflammatory effect of both individual drugs and drugs in combination on ARPE-19, ELISAs were used. Before exposing the cells to stimulants, a cytotoxicity study was carried out to identify any toxic effects of the stimulants on the cells. Based on the previous literature LPS from 0.5 to 100 μg/mL and H₂O₂ between 10 and 300 μM were tested (FIG. 4(a),(b)).

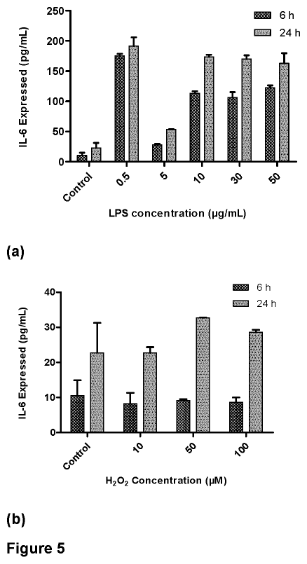

LPS proved to be non-toxic on the cells for the chosen concentrations, whereas H₂O₂ displayed a decrease in cell viability for higher concentrations (200 and 300 μM). Considering the current cytotoxicity study and the concentration used in literature, LPS between 0.5 and 50 μg/mL and H₂O₂ from 10 to 100 μM were chosen to induce inflammation (FIG. 5(a),(b)). IL-6 and IL-8 inflammatory cytokines were used to select the stimulant and the concentration to be used for further studies.

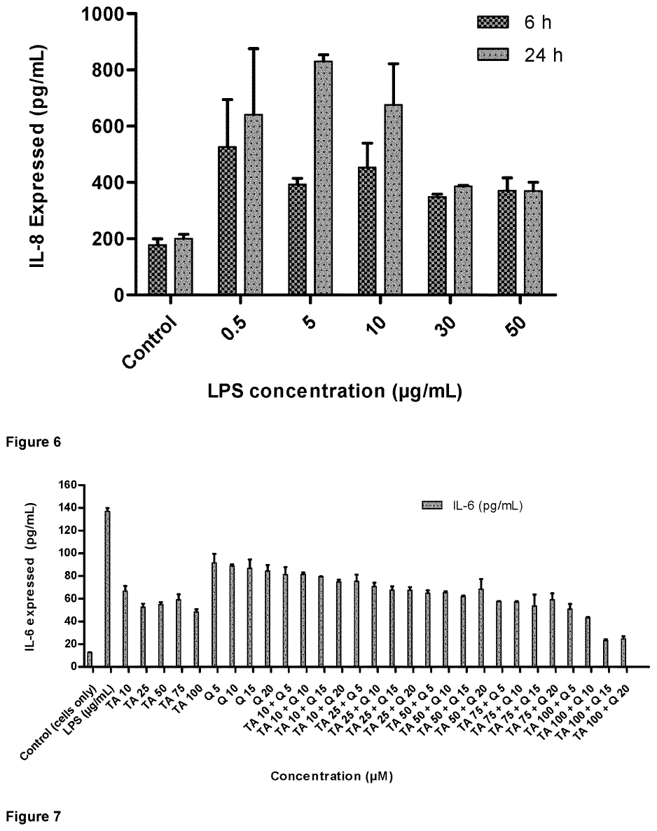

The IL-6 cytokine consistently increased with LPS stimulation from 10 to 50 g/mL but in the case of H₂O₂ the expression of IL-6 at 6 hr and 24 hr was similar to control cells. For the next screening study on IL-8 ELISA, LPS alone was investigated (FIG. 6). As expected, LPS endotoxin induced inflammation on ARPE-19 which led to the expression of IL-6 and IL-8 cytokines. When compared to the control unstimulated cells, cells stimulated with LPS showed a significant increase in the expression of cytokines with these results being in accordance with previous inflammatory studies on ARPE-19. The 10 μg/mL concentration of LPS consistently increased the expression of IL-6 and IL-8 over 24 hr period hence this condition was used for further studies. After stimulating the cells with the chosen concentration of LPS for 24 hr (except for the control unstimulated cells), cells were exposed to different concentrations of TA and QCN individually and in combination to investigate any anti-inflammatory effect (FIG. 7).

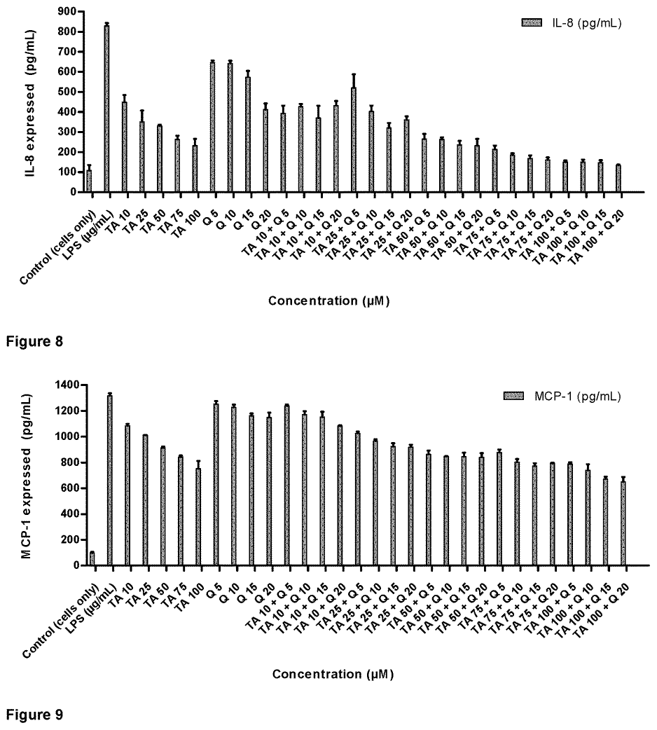

When compared to LPS stimulated cells, which expresses the maximum amount of IL-6, all the treatments exhibited a significant anti-inflammatory effect by lowering the expression of cytokine in the ELISA study. This supports the conclusion that corticosteroids like TA show an anti-inflammatory effect by multiple signal transduction pathways. In the current study TA consistently lowered the expression of IL-6 and showed maximum inhibition at 100 μM. In accordance with previous studies on ARPE-19, QCN decreased the expression of IL-6 with the maximum effect observed at 20 μM. As mentioned previously, there is no treatment for multifactorial AMD and monotherapy is not completely effective in treating the disease. In this current study, the efficacy of a novel combination of corticosteroid and flavonoid (TA+QCN) was examined. In accordance with this, different concentrations of TA+QCN were tested for their anti-inflammatory effect. When compared to individual drugs, TA and QCN displayed a synergetic decrease of IL-6 expression at higher concentrations (TA 100+QCN 15 and 20 μM) with P value >0.05. Following a similar procedure as the IL-6 ELISA study (see FIG. 6), IL-8 expression was studied with the individual drugs and in combination. All the treatments significantly reduced IL-8 cytokine expression when compared to LPS stimulated cells (FIG. 8). Whereas the higher concentrations of TA in combination with QCN (TA50+Q20, TA75+Q5, 10, 15, 20 and TA100+Q5, 10, 15, 20) showed the higher anti-inflammatory effect compared to other treatments (P<0.001). Similar to the IL-6 cytokine study of FIG. 6, a synergetic effect was observed in some combination concentrations when compared to individual drugs. TA 50 μM and 75 μM in combination with QCN 10, 15, 20 μM and TA 100 μM+Q 5 μM significantly decreased IL-8 expression and demonstrated an anti-inflammatory effect (P<0.05). The individual drugs expressed IL-8 between 232.21 and 646.46 μg/mL whereas the combination drugs treated samples saw IL-8 expression levels between 134.82 and 520.65 μg/mL. Though the difference was not statistically significant, combination treatment lowered the expression of IL-6 and IL-8 cytokines at higher concentrations of TA (75 and 100 M+QCN).

Following on from the IL-6 and IL-8 cytokines study, MCP-1 was investigated. The expression of MCP-1 cytokine was lowered for the treatments as seen in FIG. 9. Dose-dependent decrease of MCP-1 secretion was observed with increase in TA concentration from 10 to 100 μM. The higher concentrations of TA 50, 75 and 100 μM secreted MCP-1 between 750.50±61.33 to 913.36±9.78 μg/mL and in combination with QCN 15 and 20 μM the secretion was further inhibited with a range of 649.58±38.30 and 845.41±30.48 μg/mL.

### Anti-VEGF Activity

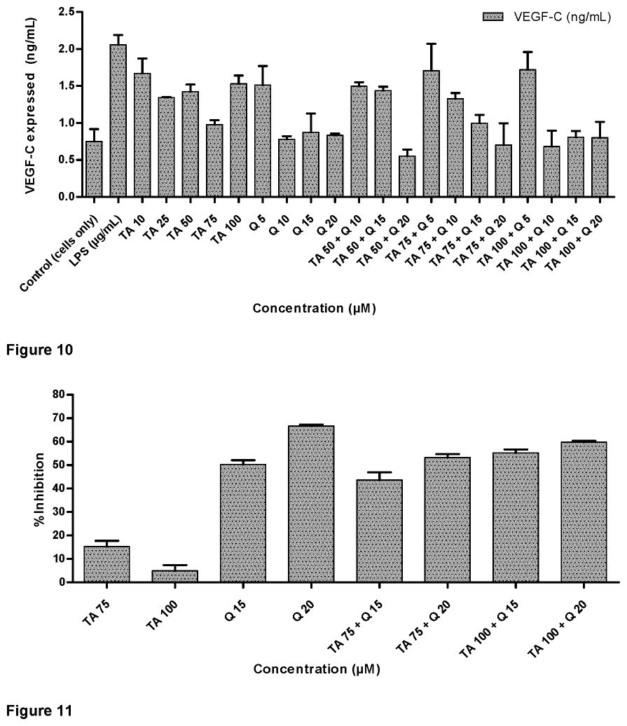

Neovascularization plays a major role in the progression of the AMD hence anti-angiogenic therapies are useful. VEGF is identified to be the major factor in promoting vascular permeability and angiogenesis. This VEGF family includes: VEGF-A, VEGF-B, VEGF-C, VEGF-D, VEGF-E and placenta growth factor (PIGF). VEGF is secreted in the ocular environment by RPE, endothelial cells and photoreceptors. Elevated levels of VEGF in the vitreous were discovered in AMD patients with neovascularization. This increase of VEGF levels will lead to damage of the blood retinal barrier, branching of blood vessels and may also stimulate inflammation by induction of inflammatory mediators like intercellular adhesion molecule-1. Due to these factors VEGF is the target for current treatments and considered as a pharmaceutical target for identifying potential treatment for AMD. Considering the findings from the current anti-inflammatory study the drug combination concentrations were filtered down and only higher concentrations of TA (75 and 100 μM) were tested in combination with higher concentrations of QCN (15 and 20 μM) on VEGF-C. The individual drugs, and combinations thereof, lowered the expression of VEGF-C when compared to LPS stimulated cells, as seen in FIG. 10. Compared to TA, QCN demonstrated better suppression of VEGF-C, by lowering the concentration from 1.96±2.05 μg/mL (control LPS stimulated) to 0.83±0.02 μg/mL (QCN 20 μM). As observed in the anti-inflammatory study, combination treatment containing higher concentrations of TA and QCN were effective.

### Antioxidant Activity

Reactive oxygen species (ROS) levels are monitored to maintain homeostasis at a cellular level. Oxidative stress is a condition where these ROS levels elevate and gather to an extent that leads to the damage of cellular macromolecules and induce apoptosis. The risk factors which lead to the progression of AMD include: genetics, aging, ethnicity and environmental factors such as high fat diet, smoking and light induced oxidative stress. Considering the role of oxidative stress in AMD, the potential therapeutics under investigation in the current study were tested to assess their antioxidant activity using DPPH and DCFH-DA assays.

The 1,1-diphenyl-2-picrylhydrazyl (DPPH) assay is one of the most commonly used colorimetric assays and gives an indication of the radical scavenging ability of the test compound. DPPH is a very stable free radical and when it comes in contact with an antioxidant it loses its free radical property resulting in a colour change from violet to yellow. The higher concentrations of TA and QCN and in combination consistently lowered inflammatory cytokine expression and VEGF-C, hence these concentrations were selected to investigate their antioxidant activity (FIG. 11). QCN efficiently decreased the level of DPPH free radical and exhibited a strong anti-oxidant effect as observed in previous studies, whereas TA displayed a minimal anti-oxidant effect. The anti-inflammatory effect of flavonoids like QCN primarily depends on their potential to scavenge ROS. This results in regulating the Nrf2 and NF-kB pathways to maintain cellular homeostasis and prevent oxidative stress. In both DPPH and DCFH-DA assays, QCN on its own and in combination demonstrated better anti-oxidant activity. QCN can regulate both enzyme-mediated and the non-enzyme-dependent antioxidant defence system. It can also regulate signal pathways such as NRFB (nuclear factor E2-related factor), AMPK (AMP-activated protein kinase), and MAPK (Mitogen-activated protein kinase) caused by ROS to promote the antioxidant defence system and maintain oxidative balance. A higher concentration of QCN (20 μM) demonstrated better anti-oxidant effect compared to 15 μM by significantly inhibiting DPPH by 66.71±0.61%. QCN 20 μM in combination with TA 75 and 100 μM displayed better radical scavenging activity than QCN 15 μM concentration.

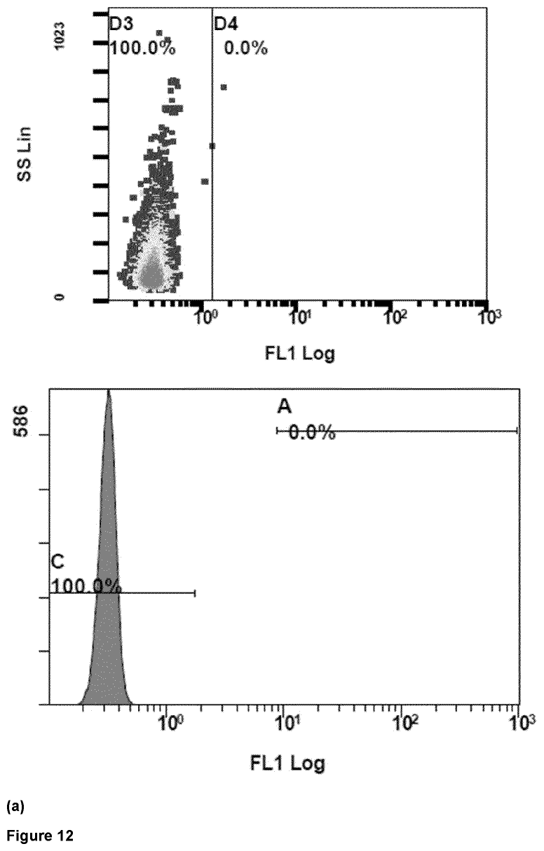

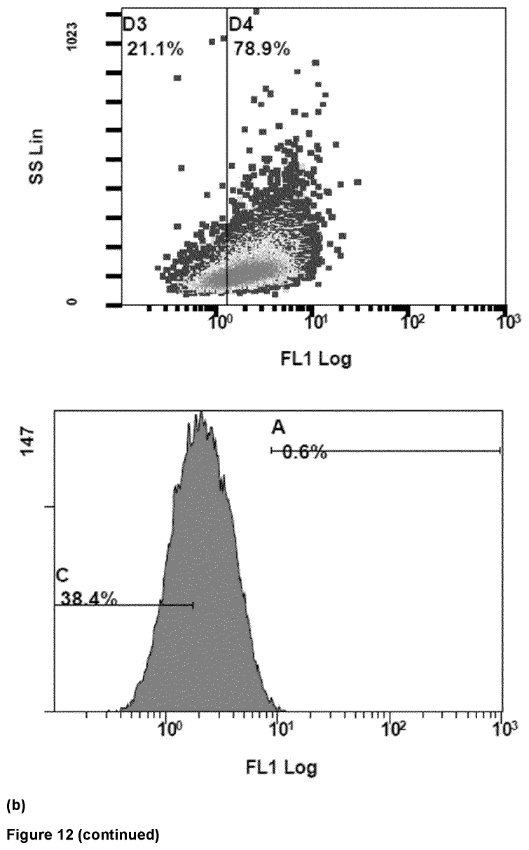

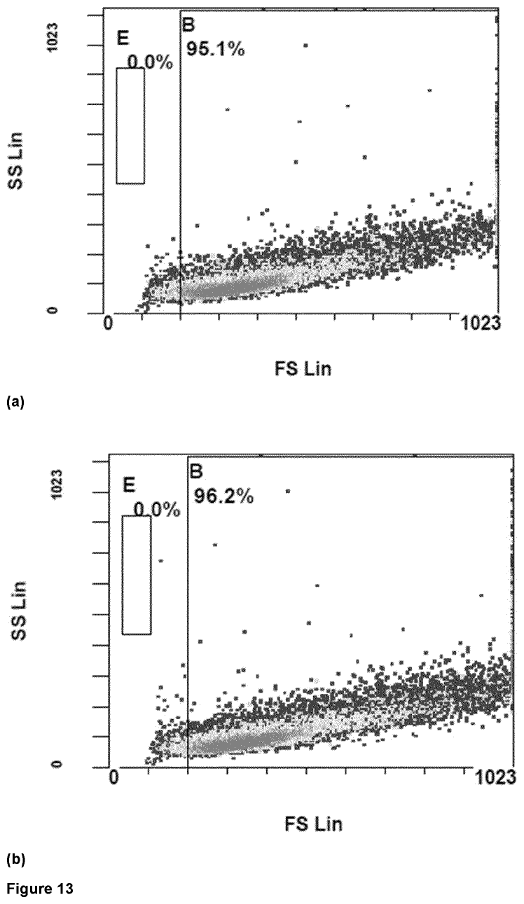

For the 2′,7′-dichlorodihydrofluorescein diacetate (DCFH-DA) assay, oxidative stress was induced to ARPE-19 cells using lipopolysaccharide and hydrogen peroxide. Upon stimulation and treatment, the intracellular ROS levels were measured using flow cytometry to assess the anti-oxidant effect of the treatments. The oxidation of DCFH-DA to 2′-7′dichlorofluorescein (DCF) was used for the detection of intracellular ROS levels including nitrogen dioxide and hydroxyl radicals. The cells will take up the DCFH-DA dye where cellular esterase cleaves off the acetyl groups, resulting in the formation of DCFH (2′,7′-dichlorodihydrofluorescein). Oxidation of DCFH in the presence of intracellular ROS leads to the formation of DCF. This DCF fluorescent molecule can be detected using a flow cytometer by mean fluorescence intensity (MFI). To induce oxidative stress, LPS and hydrogen peroxide (HP) were tested at concentrations 10, 20, 40 μg/mL and 100, 200, 300 μM, respectively. When compared to unstimulated stained and stimulated stained (with the different concentrations of stimulants), HP at 300 μM induced oxidative stress, reflected by stained cells and MFI as in FIG. 12. The ROS levels of the cells stimulated with HP at 300 μM had significantly increased when compared to unstimulated cells (MFI of unstimulated stained and stimulated stained cells was 3.73±0.29 and 2.01±0.10, respectively, with a P value <0.05). Considering the MFI value and the appearance of stained cells in the fluorescent gate, (D4) as seen in FIG. 12(b), 300 μM concentration of HP was selected for further investigation. To determine the effect of DCFH-DA dye on the morphology or the characteristics of the cells, flow cytometry analysis was performed on the control unstimulated unstained and control unstimulated stained cells. As depicted in FIG. 13, no changes of the ARPE-19 cells were observed with the exposure to the DCFH-DA dye. The forward and side scattered plots of flow cytometer analysis represent the population of ARPE-19 cells based on their size and density.

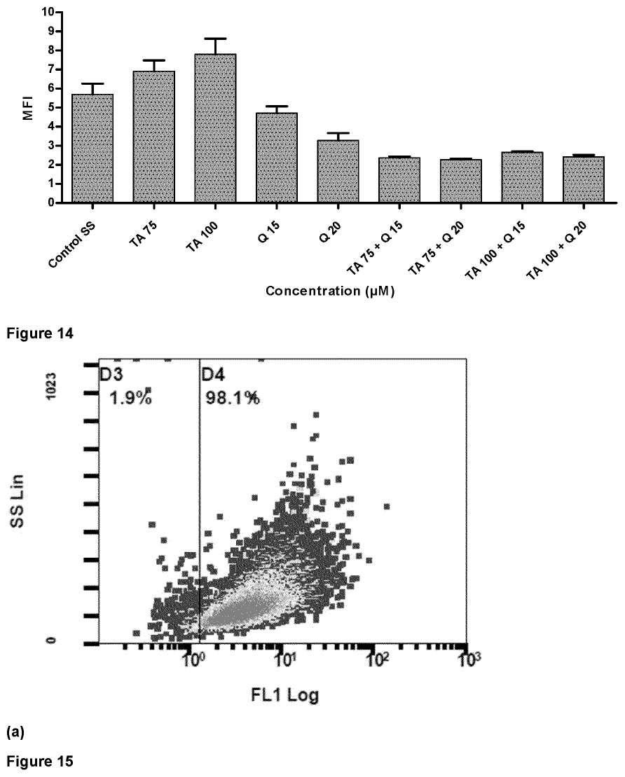

The concentrations used for the DPPH assay were those used for the investigation of the intracellular ROS levels. When compared to the control stimulated stained, QCN and all the combination concentrations significantly suppressed the generated ROS (FIG. 14). The findings of the DCFH-DA were similar to the DPPH anti-oxidant assay where TA did not lower the ROS levels. The combination drugs exhibited synergetic anti-oxidant effect by significantly reducing the ROS, which was represented by lower MFI values (P value >0.05).

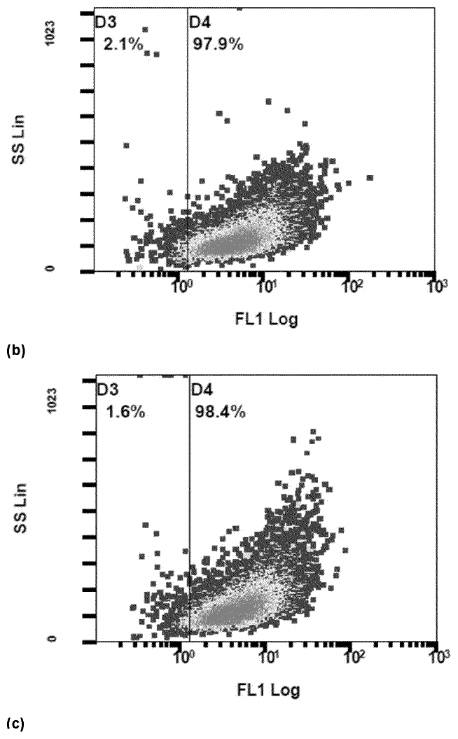

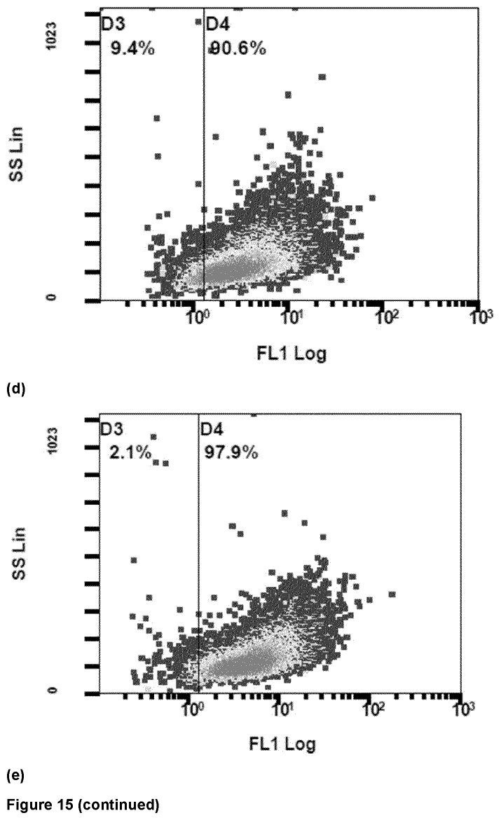

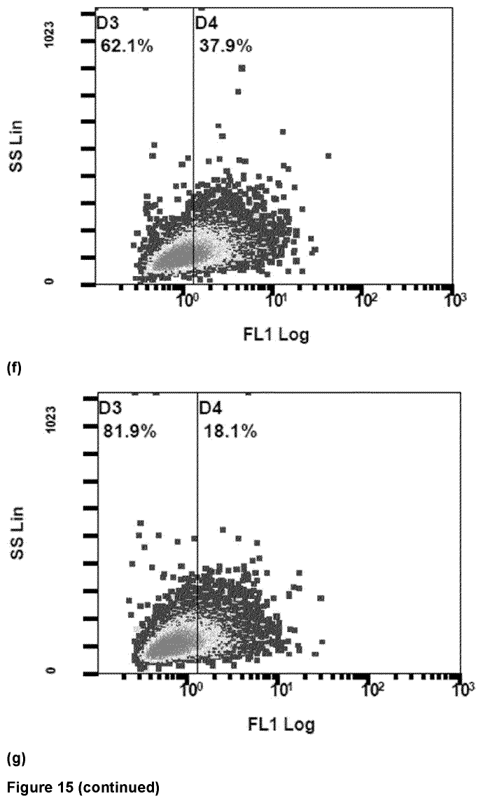

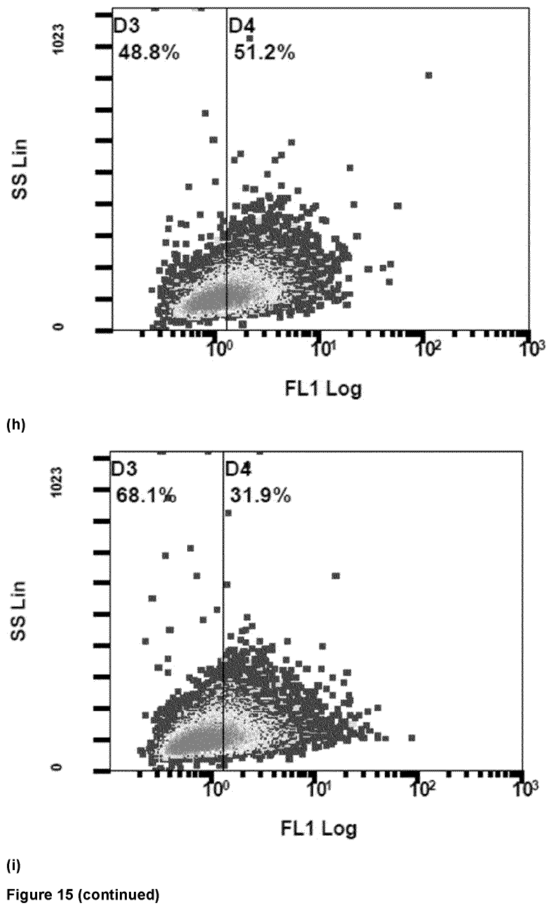

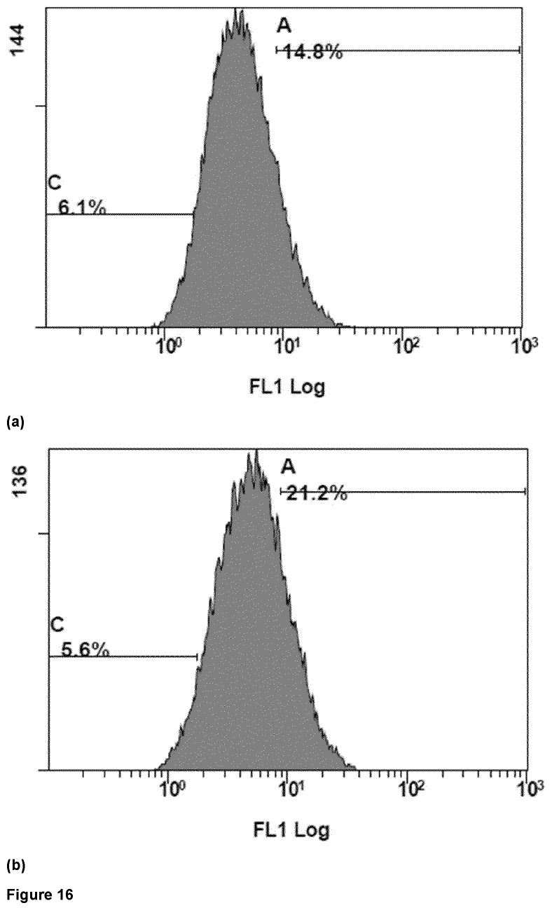

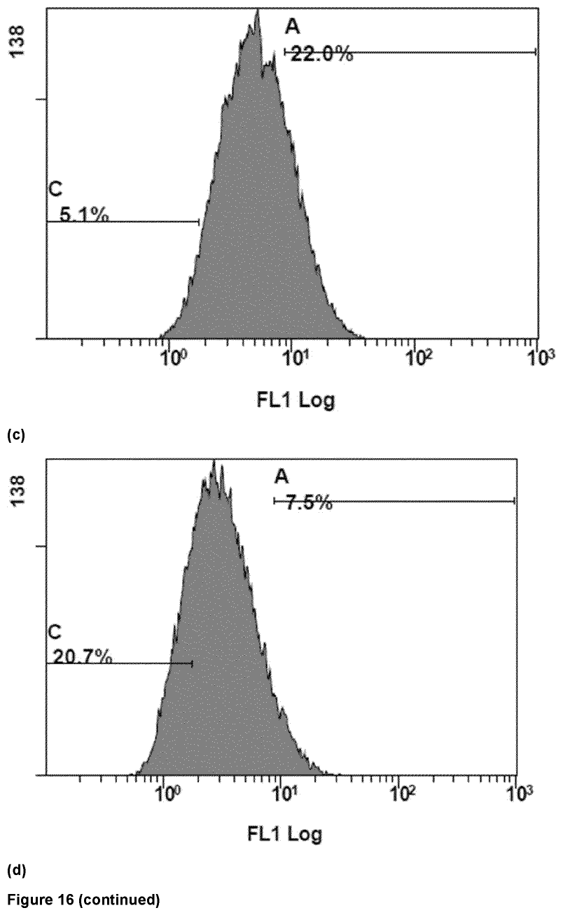

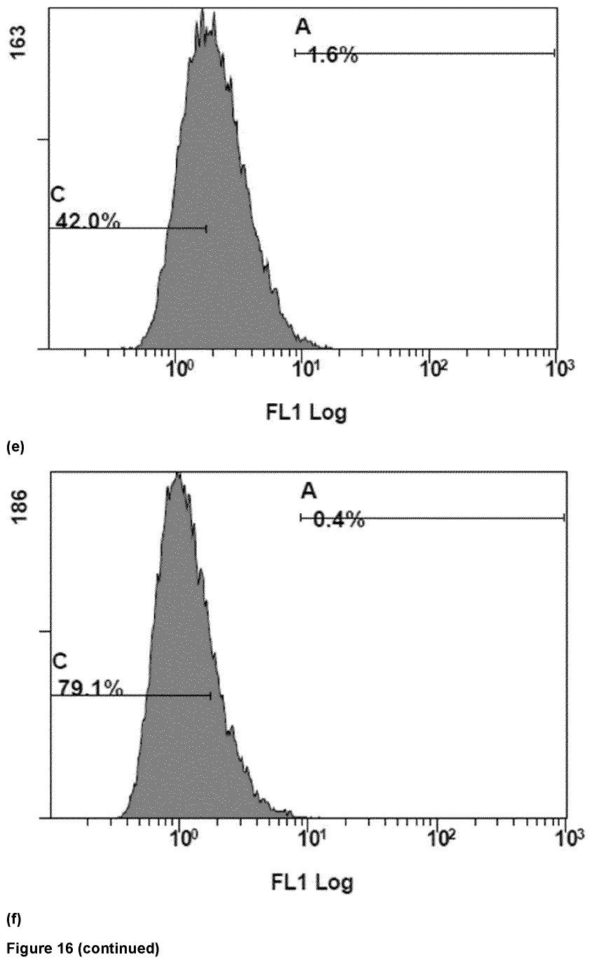

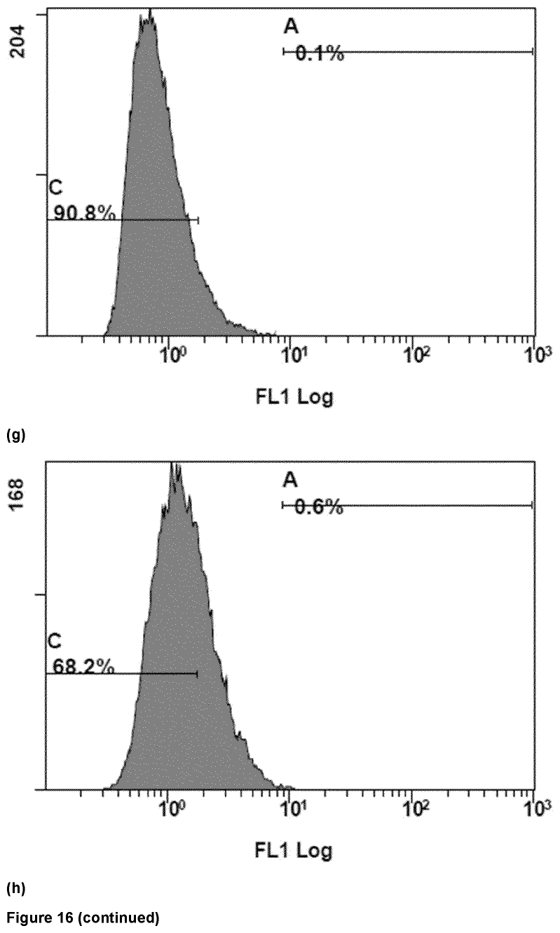

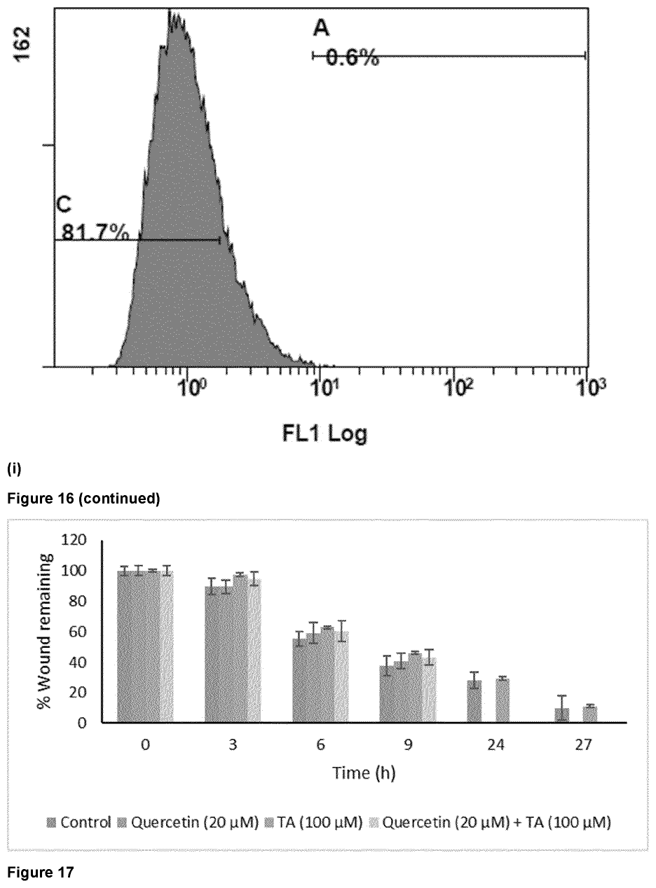

The graphs in FIG. 15 represent the stained cells with ROS in Gate D4 (fluorescent gate) and unstained cells in gate D3 (non-fluorescent gate) (the control stimulated cells have the 98.1% stained cell population in the fluorescent gate. Due to the synergetic anti-oxidant effect the stained cells containing ROS was significantly reduced, which is represented by a shift of cell population to the non-fluorescent gate. The cells containing ROS were reduced to 18.2%-51.2%, which was also represented by the increase in non-stained cells between 48.8% and 81.9%. The addition of QCN lowered the side effects of TA and enhanced the anti-oxidant activity. In accordance with the stained and unstained cell population in gates D4 and D3, the fluorescent peak shifts from 101 to 100 highlighting the strong anti-oxidant effect by reducing the intracellular ROS (FIG. 16).

### Scratch Assay

The major cause of irreversible vision loss is the exudative form of AMD, which is characterized by CNV where the newly formed blood vessels protrude from the choroid to the retina through Bruch's membrane. Even though the mechanism of CNV is not fully understood, some pathological studies point to the connection between CNV and sub-RPE deposits. Additionally, focal inflammation leads to focal thinning and damage of Bruch's membrane in patients of AMD. RPE cells are essential for the maintenance of photoreceptor cells as they provide nutrients. But the contact between RPE and photoreceptor cells was disrupted due to the accumulation of sub-RPE deposits and the interference of branched blood vessels. At this stage the migration of RPE towards the photoreceptor cells plays a vital role in protecting vision. Considering the role of migration, the scratch assay or cell migration assay was performed in the current study to assess the effect of treatments on RPE cell migration. As the scratch assay needs the cells to be fully confluent with high seeding density on 6-well plates, the concentrations were limited to higher concentration of TA and QCN and those in combination (FIG. 17). Upon seeding 25×10⁴ cells/well and reaching around 90% confluency, the wounds were created and measured using image ‘J’ software. During the study the cells were serum starved (1% FBS was used instead of 10%) to ensure the assessment of cell migration and to make sure that the wound was not closed due to cell proliferation. QCN at 20 μM and in combination with TA 100 μM enhanced the migration of the cells and wound closure was observed by the 24 hr time point (FIG. 20).

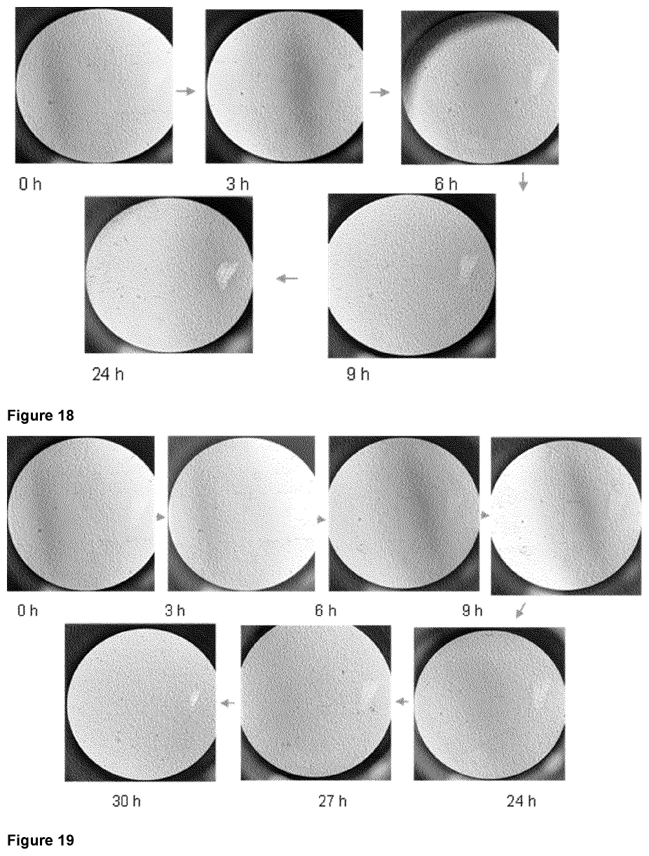

The control and TA exhibited similar pattern in the closure of the wound, TA did not enhance any cell migration (FIG. 19). During the wound creation, the ARPE-19 cells might be stressed leading to the release of ROS such as, nitric oxide due to which TA did not show any cell migration.

### CONCLUSION

Firstly, the individual drug concentrations and the combinations were safe on retinal cell line and displayed no signs of synergetic toxicity and changes in morphology of the cells. The combination exhibited a better anti-inflammatory effect as the TA and QCN act predominantly on different inflammatory signalling pathways (TA acts on NF kappa B and QCN on MAPK). In terms of anti-VEGF activity, both drugs act in a different way, QCN inhibits the kinase pathways leading to deactivation of VEGF receptors whereas TA destabilises VEGF mRNA, which lead to the greater suppression of VEGF-C with the combination treatments. Both the anti-oxidant assays (DPPH and DCFH-DA) showed similar outcomes by exhibiting the synergetic effect when treated with combination drugs (with reproducible results). QCN displayed enhanced retinal cell migration towards the wound on its own and in combination with TA. All these findings suggest corticosteroid (TA) and flavonol (QCN) as a potential combination therapeutic to target disease of the eye, for example AMD with multiple pathological conditions occurring at the same time.

In the specification the terms “comprise, comprises, comprised and comprising” or any variation thereof and the terms “include, includes, included and including” or any variation thereof are considered to be totally interchangeable and they should all be afforded the widest possible interpretation and vice versa.

The invention is not limited to the embodiments hereinbefore described but may be varied in both construction and detail.

1. A combination of agents for use in a method of treating an eye disorder in a subject, in which the combination of agents is administered to the subject and comprises a therapeutically effective dose of a corticosteroid and a flavonol.

2. The combination of agents of claim 1, for use according to claim 1, wherein the corticosteroid is selected from triamcinolone acetonide, fluocinolone acetonide, dexamethasone, prednisone, methylprednisone, cortisone, and hydrocortisone.

3. The combination of agents of claim 1 or claim 2, for use according to claim 1, wherein the flavonoid is selected from quercetin, kaempferol, and myricetin.

4. The combination of agents of any one of claims 1 to 3, for use according to claim 1, wherein the corticosteroid is triamcinolone acetonide and the flavonol is quercetin.

5. A combination of agents for use in a method of treating an eye disorder in a subject, in which the combination of agents is administered to the subject and comprises a therapeutically effective dose of a corticosteroid, triamcinolone acetonide, and a flavonol, quercetin.

6. The combination of agents of any one of the preceding claims, for use according to claim 1, wherein the concentration of the corticosteroid is selected from 1 μM to 300 μM.

7. The combination of agents of any one of the preceding claims, for use according to claim 1, wherein the concentration of the flavonol is selected from 1 μM to 75 μM.

8. The combination of agents of any one of the preceding claims, for use according to claim 1, wherein the wt % ratio of corticosteroid to flavonol in the composition is 1:0.1 to 1:1.

9. The combination of agents of claim 8, for use according to claim 1, wherein the wt % ratio of corticosteroid to flavonol in the composition is 1:0.1 to 1:0.3.

10. The combination of agents of any one of the preceding claims, for use according to claim 1, wherein the combination of agents comprises 40 μg to 50 μg of the corticosteroid and 1 μg to 10 μg of the flavonol.

11. The combination of agents of any one of the preceding claims, for use according to claim 1, wherein the corticosteroid is administered prior to administration of the flavonol.

12. The combination of agents of any one of claims 1 to 10, for use according to claim 1, wherein the corticosteroid is administered following administration of the flavonol.

13. The combination of agents of any one claims 1 to 10, for use according to claim 1, wherein the corticosteroid and the flavonol are administered simultaneously.

14. The combination of agents of one of the preceding claims, for use according to claim 1, further comprising a pharmaceutically acceptable carrier.

15. The combination of agents of one of the preceding claims, for use according to claim 1, wherein the eye disorder is selected from wet age-related macular degeneration (AMD), dry age-related macular degeneration (AMD), retinitis pigmentosa (RP), Behçet's disease, blepharitis, central retinal vein occlusion (CRVO), diabetic retinopathy, diabetic macular edema (DME), neovascular glaucoma, macular edema, uveitis, retinal vein occlusion, ocular histoplasmosis syndrome (OHS), and retinopathy of prematurity (ROP).

16. The combination of agents of claim 15, for use according to claim 1, wherein the eye disorder is wet or dry age-related macular degeneration.

17. A combination of triamcinolone acetonide and quercetin for use in a method of treating wet age-related macular degeneration (AMD) or dry age-related macular degeneration (AMD) in a subject, in which the combination of agents is administered to the subject and comprises a therapeutically effective dose of both triamcinolone acetonide and quercetin.

18. The combination of triamcinolone acetonide and quercetin of claim 17, for use according to claim 17, wherein the concentration of the triamcinolone acetonide is selected from 1 μM to 300 μM.

19. The combination of triamcinolone acetonide and quercetin according to claim 17 or claim 18, for use according to claim 1, wherein the concentration of quercetin is selected from 1 μM to 75 μM.

20. The combination of triamcinolone acetonide and quercetin of any one of claims 17 to 19, for use according to claim 17, wherein the wt % ratio of triamcinolone acetonide to quercetin to in the composition is 1:0.1 to 1:1.

21. The combination of triamcinolone acetonide and quercetin of claim 20, for use according to claim 17, wherein the wt % ratio of triamcinolone acetonide to quercetin in the composition is 1:0.1 to 1:0.3.

22. The combination of triamcinolone acetonide and quercetin of any one of claims 17 to 21, for use according to claim 17, wherein the combination of triamcinolone acetonide and quercetin comprises 40 μg to 50 μg of triamcinolone acetonide and 1 μg to 10 μg of quercetin.

23. The combination of triamcinolone acetonide and quercetin of any one of claims 17 to 22, for use according to claim 17, wherein the triamcinolone acetonide is administered prior to administration of the quercetin.

24. The combination of triamcinolone acetonide and quercetin of any one of claims 17 to 23, for use according to claim 17, wherein the triamcinolone acetonide is administered following administration of the quercetin.

25. The combination of triamcinolone acetonide and quercetin of any one claims 17 to 23, for use according to claim 17, wherein the triamcinolone acetonide and the quercetin are administered simultaneously.

26. The combination of triamcinolone acetonide of one of claims 17 to 25, for use according to claim 17, further comprising a pharmaceutically acceptable carrier.

27. A pharmaceutical composition comprising a corticosteroid and a flavonol and a pharmaceutically acceptable excipient.

28. The pharmaceutical composition according to claim 27, wherein the pharmaceutical composition is in the form of an eye drop, an intravitreal injection, a suprachoroidal injection, a retrobulbar injection, a subretinal injection, a sub-tenon injection, and a peribulbar injection.

29. A kit of parts comprising one or more doses of a corticosteroid and one or more doses of a flavonol.
